(19) United States
(12) Patent Application Publication
(10) Pub. No.: US 2002/0193773 A1
(43) Pub. Date: Dec. 19, 2002

(54) Finger-receiving area for a labial pad

(76) Inventors: Ronald L. Edens, Cumming, GA (US); James J. Hlaban, Neenah, WI (US); Laura J. Keely, Neenah, WI (US); Thomas P. Keenan, Appleton, WI (US); Sylvia B. Little, Marietta, GA (US); Mary L. McDaniel, Appleton, WI (US); Stephen L. Nunn, Appleton, WI (US); William G. Reeves, Appleton, WI (US); Heather A. Sorebo, Appleton, WI (US); Susan M. Weyenberg, Appleton, WI (US)

Correspondence Address: KIMBERLY-CLARK WORLDWIDE, INC., 401 NORTH LAKE STREET, NEENAH, WI, 54956

(21) Appl. No.: 10/039,452

(22) Filed: Dec. 31, 2001

Related U.S. Application Data
(60) Provisional application No. 60/297,000, filed on Jun. 8, 2001.

(51) Int. Cl. A61F 13/15; A61F 13/20

(52) U.S. Cl. 604/385.17; 604/385.01

(57) ABSTRACT

An absorbent article (40), such as a labial pad, configured for disposition within the vestibule (42) of a female wearer. The absorbent article (40) may be worn by females for catamenial purposes, incontinence protection, or both, and has a finger-receiving area of dimensions sufficient to receive at least a portion of at least one finger of the female wearer.

This application claims priority from U.S. Provisional Application No. 60/297,000, filed Jun. 8, 2001.

### BACKGROUND

The present invention relates generally to absorbent articles such as labial pads configured for disposition within the vestibule of a female wearer. More particularly, the present invention relates to labial pads having at least one finger-receiving area.

A broad manner and wide variety of absorbent articles configured for the absorption of bodily exudates such as menstrual fluid are, of course, well known. With respect to feminine hygiene, the art has offered two basic types of feminine hygiene protection: sanitary napkins, developed for external wear about the pudendal region, and tampons, developed for residence within the vaginal cavity and interruption of menstrual flow therefrom. Hybrid feminine hygiene protection devices, attempting to merge the structural features of both within a single type of device, have also been proposed, but have not seen a meaningful measure of acceptance insofar as the effort to appropriate advantages has been overshadowed by the more demonstrable perpetuation of structural and anatomically functional disadvantages. Other less intrusive devices, known as labial or interlabial devices and characterized as having a portion which at least partially resides external of the wearer's vestibule, have also been proposed.

Many of these prior devices have not fully satisfied the demand of consumers for even smaller devices that may be worn interlabially by female wearers. In response thereto, several manufacturers have produced labial pads that are quite small in size in comparison to the prior devices described above. However, the construction of many of these devices appears to fail to recognize the wide range of variation that exists among women with regard to the location of their vaginal and urethral orifices. For example, some current devices often locate a significant portion of the absorbent core in the center of the device with a much less significant portion of the absorbent core located in the ends. Such devices afford less protection for the broad spectrum of women whose vaginal and urethral orifices are not located in the center of their vestibules. Other devices appear to provide a uniform distribution of absorbent core substantially throughout the longitudinal length of the device. However, the designs of many of these other devices typically fail to significantly enhance individualized fit and/or absorbent capacity.

Another factor affecting consumer acceptance is the ease of use, including disposition of the labial pad into proper placement and/or removal of the labial pad. Typically, the wearer grasps the labial pad with her fingers and disposes it into a desired placement within her vestibule. The wearer may also need to grasp the labial pad for removal, particularly if it is not expelled during urination. For disposition and/or removal, it is desirable that the wearer minimize finger-contact with the body-facing surface of the labial pad. Thus, a need exists for an improved means which will facilitate sanitary disposition of a labial pad into an appropriate position within the vestibule as well as sanitary removal of a labial pad from the vestibule.

### SUMMARY

The present inventors have recognized the deficiencies and problems inherent in the prior art and in response thereto conducted intensive research in developing innovative labial pads. While conducting their research, the inventors also discovered that at least one finger-receiving area enhanced the ability of a wearer to easily dispose a labial pad within the vestibule and/or easily remove a labial pad from the vestibule.

In one embodiment, an absorbent article includes an absorbent. The absorbent article is configured for disposition within the vestibule of a female wearer. The absorbent article has a principal longitudinal axis, a principal transverse axis, a length, a width, a thickness, first and second spaced apart transverse end areas and first and second spaced apart longitudinal sides. The longitudinal sides range between the transverse end areas and collectively define a periphery of the absorbent article. The absorbent article further includes an upper surface and a lower surface. The lower surface of the absorbent article is adapted to define a finger-receiving area of dimensions sufficient to at least temporarily receive at least a portion of at least one finger of the wearer in a manner that allows at least that portion of a finger to be positioned substantially parallel to a principal axis.

Another embodiment discloses an absorbent article comprising an absorbent. The absorbent article is configured for disposition within the vestibule of a female wearer. The absorbent article has a lower surface adapted to define a finger-receiving area. The finger-receiving area has dimensions sufficient to at least temporarily receive at least a portion of at least one finger of the wearer in a manner that allows at least that portion of a finger to be positioned substantially parallel to a principal axis of the absorbent article.

A further embodiment relates to an absorbent article having an absorbent and a liquid impermeable baffle. The absorbent article is configured for disposition within the vestibule of a female wearer. The absorbent article also includes a principal longitudinal axis, a principal transverse axis, a length, a width, a thickness, first and second spaced apart transverse end areas and first and second spaced apart longitudinal sides. The longitudinal sides range between the transverse end areas and collectively define a periphery of the absorbent article. The absorbent article further includes an upper surface and a lower surface. The baffle forms at least a portion of the lower surface of the absorbent article. The lower surface of the absorbent article is adapted to define a finger-receiving area of dimensions sufficient to at least temporarily receive at least a portion of at least one finger of the wearer in a manner that allows at least that portion of a finger to be positioned substantially parallel to a principal axis.

An additional embodiment discloses an absorbent article having an absorbent and a liquid impermeable baffle. The absorbent article is configured for disposition within the vestibule of a female wearer. The absorbent also includes a lower surface. The baffle forms at least a portion of the lower surface of the absorbent article. The lower surface of the absorbent article is adapted to define a finger-receiving area of dimensions sufficient to at least temporarily receive at least a portion of at least one finger of the wearer in a manner that allows at least that portion of a finger to be positioned substantially parallel to a principal axis of the absorbent article.

An alternate embodiment relates to an absorbent article having an absorbent and a fluid permeable cover. The absorbent article is configured for disposition within the vestibule of a female wearer. The absorbent article further includes a principal longitudinal axis, a principal transverse axis, a length, a width, a thickness, first and second spaced apart transverse end areas and first and second spaced apart longitudinal sides. The longitudinal sides range between the transverse end areas and collectively define a periphery of the absorbent article. The absorbent article also includes an upper surface and a lower surface. The cover forms at least a portion of the lower surface of the absorbent article. The lower surface of the absorbent article is adapted to define a finger-receiving area of dimensions sufficient to at least temporarily receive at least a portion of at least one finger of the wearer in a manner that allows at least that portion of a finger to be positioned substantially parallel to a principal axis.

A further alternate embodiment discloses an absorbent article having an absorbent and a fluid permeable cover. The absorbent article is configured for disposition within the vestibule of a female wearer. The absorbent article also has a lower surface. The cover forms at least a portion of at least the lower surface of the absorbent article. The lower surface of the absorbent article is adapted to define a finger-receiving area of dimensions sufficient to at least temporarily receive at least a portion of at least one finger of the wearer in a manner that allows at least that portion of a finger to be positioned substantially parallel to a principal axis of the absorbent article.

### DRAWINGS

The foregoing and other features, aspects and advantages of the present invention will become better understood with regard to the following description, appended claims and accompanying drawings where:

FIG. 1 is a simplified anatomical cross-sectional view of a human female illustrating the environment for an absorbent article such as a labial pad.

FIG. 2 is a simplified anatomical cross-sectional view of a human female illustrating a placement of an absorbent article, such as a labial pad, disposed in the vestibule of a wearer.

FIG. 3 is a top view illustrating a version an absorbent article.

FIG. 4 is cross-sectional view of the version illustrated in FIG. 3 taken along line 4-4 thereof.

FIG. 5 is a cross-sectional view illustrating another version of an absorbent article.

FIG. 6 is a top view illustrating a version similar to that illustrated in FIG. 4.

FIG. 7 is a top view illustrating yet another version of an absorbent article.

FIG. 8 is a top view illustrating still another version of an absorbent article.

FIG. 9 is a top view illustrating an alternate version of an absorbent article.

FIG. 10 is a top view illustrating another alternate version of an absorbent article.

FIG. 11 is a cross-sectional view illustrating yet another alternate version of an absorbent article.

FIG. 12 is a cross-sectional view illustrating the version of FIG. 11 in a substantially folded position.

FIG. 13 illustrates a distal portion of a wearer's finger received within an embodiment of a finger-receiving area.

FIG. 14 illustrates a distal portion of a wearer's finger received within a finger-receiving area similar to the embodiment of FIG. 13.

FIG. 15 illustrates a distal portion of a wearer's finger received within another embodiment of a finger-receiving area.

FIG. 16 illustrates a distal portion of a wearer's finger received within an additional embodiment of a finger-receiving area.

FIG. 17 illustrates a distal portion of a wearer's finger received within a further embodiment of a finger-receiving area.

### DESCRIPTION

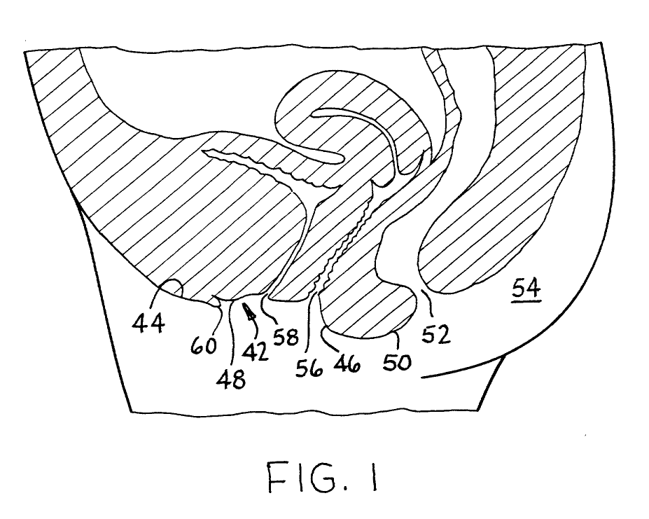

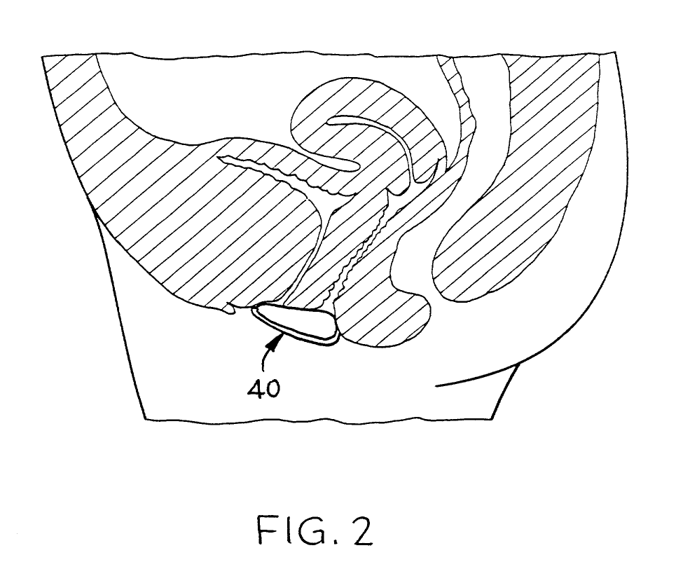

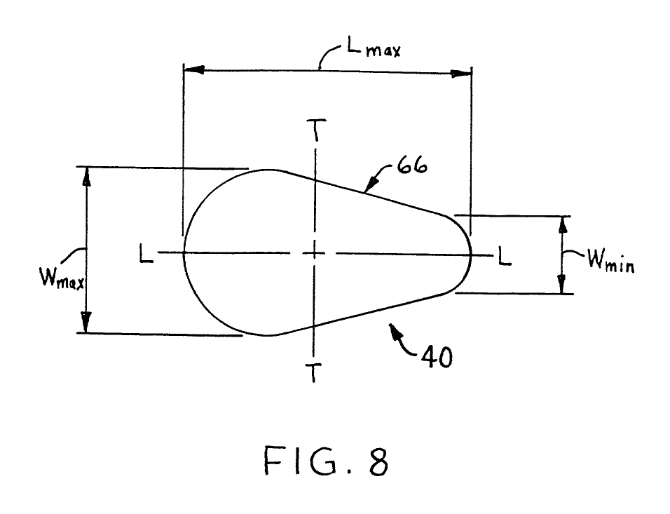

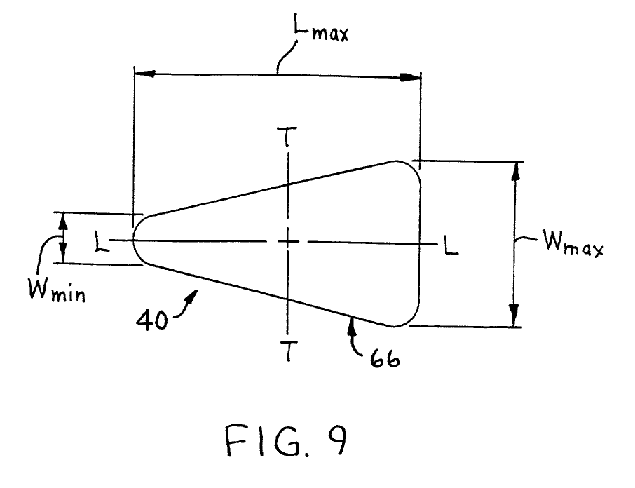

Turning to the figures of drawing, i.e., FIGS. 1 through 17, in each of which similar parts are identified with like reference characters, FIG. 2 illustrates diagrammatically an absorbent article, such as a labial pad, designated generally as 40, disposed within the vestibule of a wearer, designated generally as 42 (see also FIG. 1). As used herein, the term “labial pad” refers to a device having at least some absorbent components, and which is specifically configured for disposition in between the labia majora, extending at least partially into the vestibule (42) of a female wearer during use. For purposes of the ensuing description, the vestibule (42) is considered to be the region defined within the labia (not specifically shown in the figures herein) beginning at about a point lying caudally from the anterior labial commissure (44), extending rearward to the posterior labial commissure (46) and bounded inwardly by the floor (48) of the vestibule. One of skill in the art fully understands that there is a wide range of variation among women with respect to the relative size and shape of labia majora and labia minora as the same interrelatedly define the contour of the vestibule (42). For purposes of the present description, however, such differences will not specifically be addressed, it being recognized that in any event the disposition of the absorbent article (40) into the vestibule (42) will necessitate placement between the labia majora regardless of any such consideration respecting the labia minora. Lying caudally of the vestibule (42) is the perineum (50) which leads to the anus (52) in the region of the buttocks (54). Within the vestibule (42) itself is located the principal urogenital members which, for purposes pertinent here, are constituted of the vaginal orifice (56), the urethral orifice (58), and the clitoris (60). Given the foregoing simplified review of this anatomical region, and to facilitate the present description, the vestibule (42) will be considered generally to be the region between the posterior labial commissure (46) and the clitoris (60), for convenience. For a more comprehensive description of this portion of the human female anatomy, however, attention is invited to Anatomy of the Human Body by Henry Gray, Thirtieth American Edition (Carmine D. Clemente ed., Lea & Febiger, 1985) at 1571-1581.

As can be seen with reference to the anatomical structure illustrated in FIGS. 1 and 2, the absorbent article (40) is disposed at least partially within the vestibule (42) for at least partially occluding the same respecting fluid flow therefrom. In this regard, the predominant use of the absorbent article (40) is for the absorption of menstrual fluid emitted via the vaginal orifice (56); although the absorbent article is equally well adapted to serve as a type of incontinence device for absorption of urine as occurs upon minor, female incontinence.

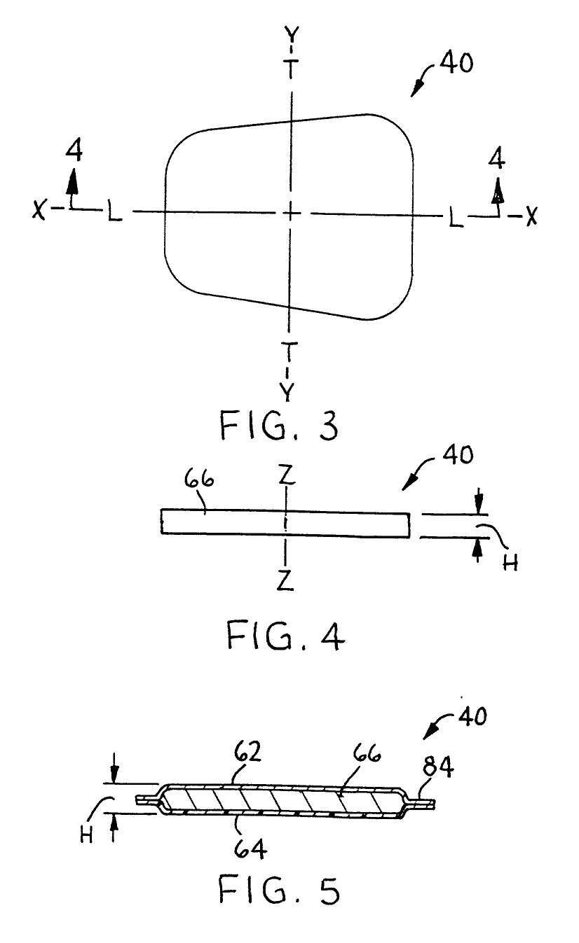

The absorbent article (40), a version of which is illustrated in FIG. 3, has a principal longitudinal axis (L) which generally runs along the x direction. As used herein, the term “longitudinal” refers to a line, axis or direction in the plane of the absorbent article (40) that is generally aligned with (e.g., approximately parallel to) a vertical plane that bisects a standing female wearer into left and right body halves when the absorbent article is in use. The longitudinal direction is generally illustrated in FIG. 3 by the x-axis. The absorbent article (40) also has a principal transverse axis (T). The terms “transverse,” “lateral” or “y direction” as used herein generally refer to a line, axis or direction that is generally perpendicular to the longitudinal direction. The lateral direction is generally illustrated in FIG. 3 by the y-axis. The “z direction” is typically a line, axis or direction generally parallel to the vertical plane described above. The z direction is generally illustrated in FIG. 4 by the z-axis. The term “upper” refers generally to an orientation directed toward the wearer's head, while the terms “lower” or “downwardly” refer generally to an orientation directed toward the wearer's feet. For purposes of discussion herein, each layer of the absorbent article (40), e.g., a fluid permeable cover (62), a liquid impermeable baffle (64) and/or an absorbent (66), has an upper or body-facing surface and a lower surface also described as the surface opposed to the upper or body-facing surface.

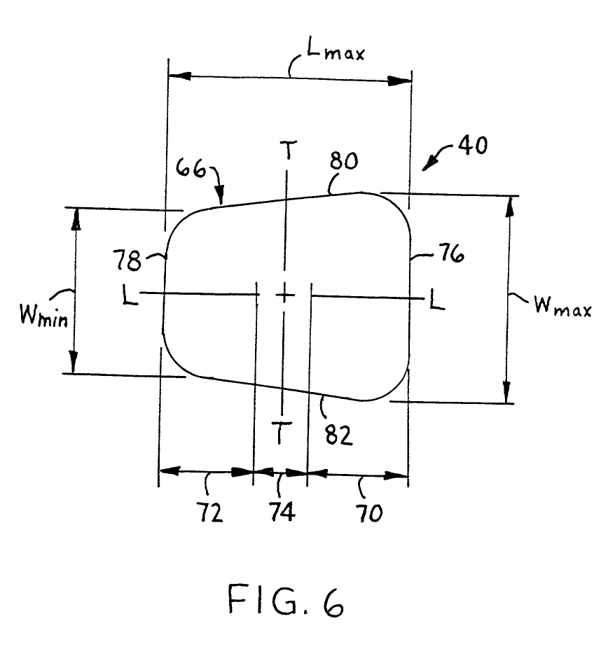

Turning now to FIG. 5, an absorbent article (40) is illustrated as including a fluid permeable cover (62), a liquid impermeable baffle (64) and an absorbent (66) situated between the cover and the baffle. As illustrated in FIG. 6, the absorbent (66) has a first end region (70), a second end region (72), and a central region (74) disposed between each end region. The absorbent article (40) should be of a suitable size and shape that allows at least a portion of the absorbent article to be disposed within the vestibule (42) of a female wearer. In addition, the absorbent article (40) desirably at least partially occludes and intercepts the flow of menstrual fluid, urine or other bodily exudates from the wearer's vaginal orifice (56) and/or urethral orifice (58).

The absorbent (66), and thus the absorbent article (40), generally displays a geometry extending between spaced apart first (76) and second (78) transverse end areas. The overall geometry is completed by noting that the absorbent (66), and thus the absorbent article (40), also includes spaced apart first (80) and second (82) longitudinal sides ranging between the transverse end areas (76, 78), these collectively sometimes being referred to herein as the perimetral sides (i.e., those defining the periphery).

The geometry of the absorbent (66) is a significant factor affecting the overall size and effectiveness of the absorbent article (40). In general, the absorbent (66) has a maximum width (Wₘₐₓ), measured along a line laying generally parallel to the principal transverse axis (T) and running from one longitudinal side to the opposing longitudinal side (80, 82), and a minimum width (Wₘᵢₙ), measured along a line also laying generally parallel to the principal transverse axis (T) and running from one longitudinal side to the opposing longitudinal side (80, 82). The maximum width (Wₘₐₓ) of the absorbent (66) typically is no greater than about 30; alternatively, no greater than about 40; alternatively, no greater than about 50; alternatively, no greater than about 60; or alternatively, no greater than about 70 mm. The minimum width (Wₘᵢₙ) of the absorbent (66) typically is no less than about 30; alternatively, no less than about 20; alternatively, no less than about 10; or alternatively, no less than about 5 mm. Thus, the absorbent (66) may have a width ranging between no less than about 5 mm up to no greater than about 70 mm; although the approximate width(s) of the absorbent may vary according to, inter alia, the general design and intended disposition of the absorbent article (40) within the vestibule (42) of a female wearer. One of skill in the art will readily appreciate that certain versions of the absorbent (66), and thus certain versions of the absorbent article (40), may have a minimum width (Wₘᵢₙ) equal to its maximum width (Wₘₐₓ). In such instances, reference is generally made only to the maximum width (Wₘₐₓ).

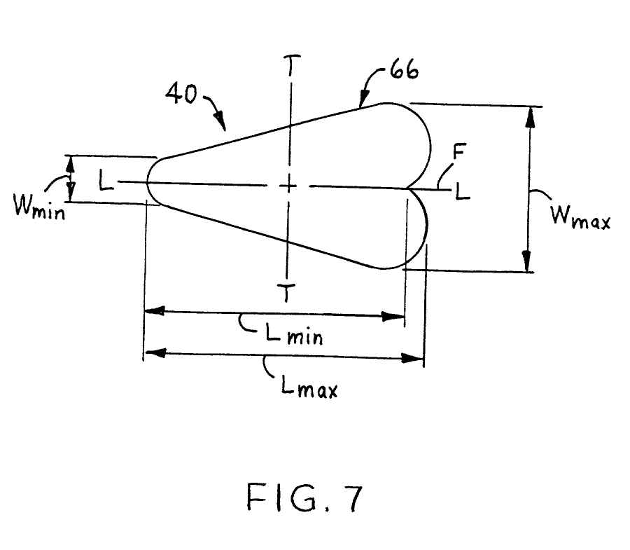

The absorbent (66) also has a maximum length (Lₘₐₓ), measured along a line laying generally parallel to the principal longitudinal axis (L) and running from one transverse end area to the other transverse end area (76, 78). The maximum length (Lₘₐₓ) of the absorbent (66) typically is no greater than about 40; alternatively, no greater than about 50; alternatively, no greater than about 60; alternatively, no greater than about 70; alternatively, no greater than about 80; alternatively, no greater than about 90; or alternatively, no greater than about 100 mm. The absorbent (66) may also have a minimum length (Lₘᵢₙ), measured along a line also laying generally parallel to the principal longitudinal axis (L) and running from one transverse end area to the other transverse end area (76, 78). The minimum length (Lₘᵢₙ) of the absorbent (66) typically is no less than about 100; alternatively, no less than about 90; alternatively, no less than about 80; alternatively, no less than about 70; alternatively, no less than about 60; alternatively, no less than about 50; or alternatively, no less than about 40 mm. Thus, the absorbent (66) may have a length ranging between no less than about 40 mm up to no greater than about 100 mm; although the approximate length(s) of the absorbent may vary according to, inter alia, the general design and intended disposition of the absorbent article (40) within the vestibule (42) of a female wearer. One of skill in the art will readily appreciate that certain versions of the absorbent (66), and thus certain versions of the absorbent article (40), may have a minimum length (Lₘᵢₙ) equal to its maximum length (Lₘₐₓ) In such instances, as illustrated at least in FIG. 6, reference is generally made only to the maximum length (Lₘₐₓ). A version of an absorbent (66), and thus a version of an absorbent article (40), having a maximum length (Lₘₐₓ) not equal to its minimum length (Lₘᵢₙ) is illustrated at least in FIG. 7.

The first end region (70) and the second end region (72) each minimally extend outwardly from the central region (74) toward the transverse end areas (76 and 78, respectively) of the absorbent (66) a distance of no less than about 30; alternatively, no less than about 20; or alternatively, no less than about 10% of the maximum length (Lₘₐₓ) of the absorbent. The first end region (70) and the second end region (72) each maximally extend outwardly from the central region (74) toward the transverse end areas (76 and 78, respectively) of the absorbent (66) a distance of no greater than about 20; alternatively, no greater than about 30; or alternatively, no greater than about 40% of the maximum length (Lₘₐₓ) of the absorbent. Thus, the end regions (70, 72) may occupy from a minimum of about 20% up to a maximum of about 80% of the maximum length (Lₘₐₓ) of the absorbent (66); although the approximate size of the first and second end regions may vary according to, inter alia, the general design and intended disposition of the absorbent article (40) within the vestibule (42) of a female wearer.

The absorbent article (40) is desirably provided with sufficient capacity to absorb and retain the intended amount and type of bodily exudate(s). The absorbent capacity is provided by a fluid retentive core or absorbent generally identified as 66. For at least menstrual fluid, the absorbent (66) desirably has a minimum capacity of no less than about 19; alternatively, no less than about 18; alternatively, no less than about 17; alternatively, no less than about 16; alternatively, no less than about 15; alternatively, no less than about 14; alternatively, no less than about 13; alternatively, no less than about 12; alternatively, no less than about 11; alternatively, no less than about 10; alternatively, no less than about 9; alternatively, no less than about 8; alternatively, no less than about 7; alternatively, no less than about 6; alternatively, no less than about 5; alternatively, no less than about 4; alternatively, no less than about 3; alternatively, no less than about 2; or alternatively, no less than about 1 g/g. The absorbent (66) also may have a maximum capacity of no greater than about 5; alternatively, no greater than about 6; alternatively, no greater than about 7; alternatively, no greater than about 8; alternatively, no greater than about 9; alternatively, no greater than about 10; alternatively, no greater than about 11; alternatively, no greater than about 12; alternatively, no greater than about 13; alternatively, no greater than about 14; alternatively, no greater than about 15; alternatively, no greater than about 16; alternatively, no greater than about 17; alternatively, no greater than about 18; alternatively, no greater than about 19; alternatively, no greater than about 20; alternatively, no greater than about 25; or alternatively, no greater than about 30 g/g. Thus, the absorbent (66) may have an absorbent capacity ranging between no less than about 1 g/g up to no greater than about 30 g/g; although the approximate capacity of the absorbent may vary according to, inter alia, the general design and intended disposition of the absorbent article (40) within the vestibule (42) of a female wearer. One of skill in the art will readily realize that the addition of superabsorbent polymer(s) or coated superabsorbent polymer(s) to the absorbent (66) typically has the effect of substantially increasing the absorbent capacity.

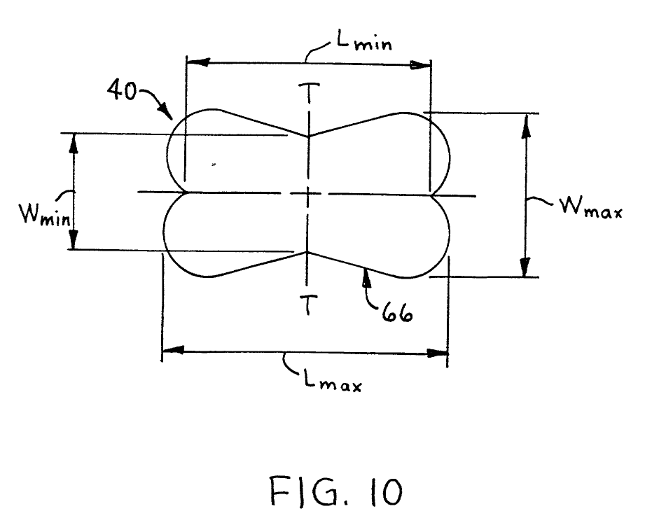

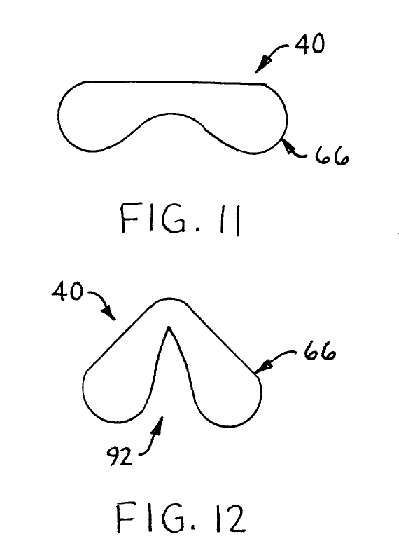

Describing the individual elements in greater detail, the absorbent (66) has an upper or body-facing surface and a lower surface (or surface opposed to the upper or body-facing surface) and may include any material capable of absorbing and/or adsorbing and thereafter retaining the intended bodily exudate(s). Suitable materials are also generally hydrophilic, compressible and conformable. The absorbent (66) may be formed from any of the materials well known to those of ordinary skill in the art. Examples of such materials include, but are not limited to, various natural or synthetic fibers, multiple plies of creped cellulose wadding, fluffed cellulose fibers, rayon or other regenerated cellulose materials, wood pulp fibers or comminuted wood pulp fibers, airlaid material, textile fibers, a blend of polyester and polypropylene fibers, absorbent foams, absorbent sponges, superabsorbent polymers, coated superabsorbent polymers, fibrous bundles or nits, or any equivalent material or combination of materials. Also suitable for use would be hydrophobic material that has been rendered hydrophilic according to any of a number of known methods for so doing. The total absorbent capacity of the absorbent (66) should, however, be compatible with the design exudate loading and the intended use of the absorbent article (40). Further, the size and absorbent capacity of the absorbent (66) may be varied. Therefore, the dimension, shape, and configuration of the absorbent (66) may be varied (e.g., the absorbent may have a varying thickness as illustrated at least in FIGS. 11 and 12, or a hydrophilic gradient, or may contain superabsorbent polymer(s) and the like).

The absorbent (66) generally has a thickness, caliper or height (H), as illustrated at least in FIG. 4, measured along a line lying generally parallel to the z-axis. The minimum thickness of the absorbent (66) typically is no less than about 9; alternatively, no less than about 8; alternatively, no less than about 7; alternatively, no less than about 6; alternatively, no less than about 5; alternatively, no less than about 4; alternatively, no less than about 3; alternatively, no less than about 2; alternatively, no less than about 1; or alternatively, no less than about 0.5 mm. The maximum thickness of the absorbent (66) typically is no greater than about 2; alternatively, no greater than about 3; alternatively, no greater than about 4; alternatively, no greater than about 5; alternatively, no greater than about 6; alternatively, no greater than about 7; alternatively, no greater than about 8; alternatively, no greater than about 9; or alternatively, no greater than about 10 mm. Thus, the absorbent (66) may have a thickness of about 10 mm or less; although the approximate thickness of the absorbent may vary according to, inter alia, the general design and intended disposition of the absorbent article (40) within the vestibule (42) of a female wearer.

The absorbent (66) desirably also has a relatively low density which is deemed desirable for comfort. Generally, the absorbent has a density of less than about 0.5 g/cc. Stated differently, the absorbent (66) typically has a maximum density of no greater than about 0.5; alternatively, no greater than about 0.4; alternatively, no greater than about 0.3; alternatively, no greater than about 0.2; alternatively, no greater than about 0.1; alternatively, no greater than about 0.09; alternatively, no greater than about 0.08; alternatively, no greater than about 0.07; alternatively, no greater than about 0.06; alternatively, no greater than about 0.05; alternatively, no greater than about 0.04; alternatively, no greater than about 0.03; or alternatively, no greater than about 0.02 g/cc. The absorbent (66) generally also has a minimum density of typically no less than about 0.01; alternatively no less than about 0.02; alternatively, no less than about 0.03; alternatively, no less than about 0.04; alternatively, no less than about 0.05; alternatively, no less than about 0.06; alternatively, no less than about 0.07; alternatively, no less than about 0.08; alternatively, no less than about 0.09; alternatively, no less than about 0.1; alternatively, no less than about 0.2; alternatively, no less than about 0.3; or alternatively, no less than about 0.4 g/cc. Thus, the density of the absorbent (66) may range up to about 0.5 g/cc; although the approximate density of the absorbent may vary according to, inter alia, the general design and intended disposition of the absorbent article (40) within the vestibule (42) of a female wearer.

The absorbent (66) also desirably has a basis weight of less than about 600 grams per square meter (gsm). Stated differently, the absorbent (66) typically has a maximum basis weight of no greater than about 600; alternatively, no greater than about 500; alternatively, no greater than about 400; alternatively, no greater than about 300; alternatively, no greater than about 200; or alternatively, no greater than about 100 gsm. Generally, the absorbent (66) also has a minimum basis weight of typically no less than about 0.1; alternatively, no less than about 50; alternatively, no less than about 100; alternatively, no less than about 150; alternatively, no less than about 200; alternatively, no less than about 250; alternatively, no less than about 300; alternatively, no less than about 350; alternatively, no less than about 400; alternatively, no less than about 450; alternatively, no less than about 500; or alternatively, no less than about 550 gsm. Thus, the absorbent (66) may have a basis weight of about 600 gsm or less; although the approximate basis weight of the absorbent may vary according to, inter alia, the general design and intended disposition of the absorbent article (40) within the vestibule (42) of a female wearer. A specific example of a suitable absorbent would be similar to a coform material made of a blend of polypropylene and cellulose fibers and used in KOTEX® maxi pantiliners and obtainable from Kimberly-Clark Corporation, Neenah, Wis., USA.

The optional baffle (64) typically resides on the lower surface of the absorbent (66) and may be constructed from any desired material that is liquid-impermeable. Desirably, the baffle (64) will permit the passage of air and moisture vapor out of the absorbent (66), while blocking the passage of bodily fluid(s). An example of a suitable baffle material is a micro-embossed, polymeric film, such as polyethylene, polypropylene or polyester, having a minimum thickness of no less than about 0.025 mm and a maximum thickness of no greater than about 0.13 mm. Bicomponent films can also be used, as well as woven and nonwoven fabrics which have been treated to render them liquid-impermeable. An example of another suitable material is a closed cell polyolefin foam. A closed cell polyethylene foam may also work well.

The baffle (64) may be maintained in secured relation with the absorbent (66) by bonding all or a portion of the adjacent surfaces to one another. A variety of bonding methods known to one of skill in the art may be utilized to achieve any such secured relation. Examples of such methods include, but are not limited to, ultrasonics, thermal bonding, or the application of adhesives in a variety of patterns between the two adjoining surfaces. A specific example of a baffle material would be similar to a polyethylene film used on KOTEX® pantiliners and obtainable from Pliant Corporation, Schaumburg, Ill., USA.

The optional fluid permeable cover (62) has an upper surface and a lower surface, with the upper surface typically contacting the body of the wearer and receiving bodily exudate(s). The cover (62) desirably is made of a material that is flexible and non-irritating to the tissues within the vestibule (42) of a female wearer. As used herein, the term “flexible” is intended to refer to materials which are compliant and readily conform to the bodily surface(s) or respond by easily deforming in the presence of external forces.

The cover (62) is provided for comfort and conformability and functions to direct bodily exudate(s) away from the body and toward the absorbent (66). The cover (62) should retain little or no liquid in its structure so that it provides a relatively comfortable and non-irritating surface next to the tissues within the vestibule (42) of a female wearer. The cover (62) can be constructed of any woven or nonwoven material which is also easily penetrated by bodily fluids contacting its surface. Examples of suitable materials include rayon, bonded carded webs of polyester, polypropylene, polyethylene, nylon, or other heat-bondable fibers, polyolefins, such as copolymers of polypropylene and polyethylene, linear low-density polyethylene, aliphatic esters such as polylactic acid, finely perforated film webs and net material also work well. A specific example of a suitable cover material would be similar to a bonded carded web made of polypropylene and polyethylene used as a cover stock for KOTEX pantiliners and obtainable from Sandler Corporation, Germany. Other examples of suitable materials are composite materials of a polymer and a nonwoven fabric material. The composite materials are typically in the form of integral sheets generally formed by the extrusion of a polymer onto a web of spunbond material. The fluid permeable cover (62) can also contain a plurality of apertures (not shown) formed therein which are intended to increase the rate at which bodily fluid(s) can penetrate into the absorbent (66).

A physiologically hydrous cover material is also suitable for use. As used herein, the term “physiologically hydrous” is intended to connote a cover material which maintains a suitably moist interface between the tissues of the vestibule (42) and the absorbent article (40) when disposed in that vestibular environment; one that is benign respecting the requirements of comfort associated with the interposition of fabric or fabric-like structures within the moist tissue environment of the vestibule, keeping in mind as well the self-evident factor that the absorbent article is receiving bodily fluid(s) migrating through the vestibule and must conduct the same to the absorbent (66). Thus, while not “hydrous” in the classic sense prior to use (inasmuch as the cover will be dry at that time) the cover (62) maintains (or at least does not interfere with the maintenance of) the proper moisture level or balance required within the vestibule (42).

The cover (62) can also have at least a portion of the surface treated with a surfactant to render the cover more hydrophilic. This results in permitting the insulting bodily fluid(s) to more readily penetrate the cover (62). The surfactant may also diminish the likelihood that the insulting bodily fluid(s), such as menstrual fluid, will flow off the cover (62) rather than being absorbed by the absorbent (66). One suitable approach provides for the surfactant to be substantially evenly distributed across at least a portion of the upper surface of the cover (62) that overlays the upper surface of the absorbent (66).

The cover (62) may be maintained in secured relation with the absorbent (66) by bonding all or a portion of the adjacent surfaces to one another. A variety of bonding methods known to one of skill in the art may be utilized to achieve any such secured relation. Examples of such methods include, but are not limited to, the application of adhesives in a variety of patterns between the two adjoining surfaces, entangling at least portions of the adjacent surface of the absorbent with portions of the adjacent surface of the cover, or fusing at least portions of the adjacent surface of the cover to portions of the adjacent surface of the absorbent.

The cover (62) typically resides on the upper surface of the absorbent (66), but alternatively can surround and partially or entirely enclose the absorbent. Alternatively, the cover (62) and the baffle (64) can have peripheries which extend outward beyond the periphery of the absorbent (66) and can be peripherally joined together to form an edge (84), as illustrated at least in FIG. 5. Utilizing known techniques, such as, for example, gluing, crimping, hot-sealing or the like, the edge (84) may be formed either entirely, so that the entire periphery of the absorbent (66) is circumscribed by their joinder, or the cover (62) and the baffle (64) can be partially peripherally joined. To minimize the possibility of irritation and/or discomfort to the wearer of the absorbent article (40), it is desired that the edge (84) and at least the area of the absorbent article immediately adjacent the edge be soft, compressible and conformable. Desirably, any edge (84) so formed shall have a width no greater than about 10; alternatively, no greater than about 9; alternatively, no greater than about 8; alternatively, no greater than about 7; alternatively, no greater than about 6; alternatively, no greater than about 5; alternatively, no greater than about 4; alternatively, no greater than about 3; alternatively, no greater than about 2; or alternatively, no greater than about 1 mm. In addition, any edge (84) so formed shall desirably have a width of no less than about 0.5; alternatively, no less than about 1; alternatively, no less than about 2; alternatively, no less than about 3; alternatively, no less than about 4; alternatively, no less than about 5; alternatively, no less than about 6; alternatively, no less than about 7; alternatively, no less than about 8; or alternatively, no less than about 9 mm. Thus, any edge (84) so formed may have a width ranging from no less than about 0.5 mm up to no greater than about 10 mm; although the approximate width of any edge may vary according to, inter alia, the general design and intended disposition of the absorbent article (40) within the vestibule (42) of a female wearer. In other versions, the cover (62) and/or the baffle (64) can have a periphery that is coterminous with the periphery of the absorbent (66).

Positioned either on or substantially parallel to the principal longitudinal axis (L) of the absorbent (66), is, optionally, a desired axis of flexure (F). A desired axis of flexure (F) generally runs in the longitudinal direction, i.e., along the x direction, and may be off center from the principal longitudinal axis (L) a distance of no greater than about 10; alternatively, no greater than about 9; alternatively, no greater than about 8; alternatively, no greater than about 7; alternatively, no greater than about 6; alternatively, no greater than about 5; alternatively, no greater than about 4; alternatively, no greater than about 3; alternatively, no greater than about 2; or alternatively, no greater than about 1 mm. Desirably, a desired axis of flexure (F) is aligned along the principal longitudinal axis (L). A desired axis of flexure (F) typically minimally extends longitudinally no less than about 90; alternatively, no less than about 80; alternatively, no less than about 70; alternatively, no less than about 60; alternatively, no less than about 50; or alternatively, no less than about 40% of the maximum length (Lₘₐₓ) of the absorbent (66). A desired axis of flexure (F) typically extends longitudinally no greater than about 50; alternatively, no greater than about 60; alternatively, no greater than about 70; alternatively, no greater than about 80; alternatively, no greater than about 90; or alternatively, no greater than about 100% of the maximum length (Lₘₐₓ) of the absorbent (66). A desired axis of flexure (F) may result naturally from the dimensions, shape, and/or configuration of the absorbent (66), or the absorbent may be imparted with a weakened axis or region to create a desired axis of flexure. A desired axis of flexure (F) may also be formed by any of the techniques known to one of skill in the art, including, for example, scoring, pre-folding, slitting, embossing, or the like. Although a desired axis of flexure (F) is described herein as residing in the absorbent (66), one of skill in the art will readily appreciate that a desired axis of flexure may be formed in either the cover (62), the baffle (64) and/or the absorbent; the cover and the baffle; the cover and the absorbent; or the baffle and the absorbent. When present, a desired axis of flexure (F) typically allows an absorbent article (40) to be folded more easily prior to disposition within the vestibule (42) of a female wearer.

The absorbent article (40) also has a thickness, caliper or height (H), as illustrated at least in FIGS. 4 and 5, measured along a line laying generally parallel to the z-axis. The minimum thickness of the absorbent article (40) typically is no less than about 9; alternatively, no less than about 8; alternatively, no less than about 7; alternatively, no less than about 6; alternatively, no less than about 5; alternatively, no less than about 4; alternatively, no less than about 3; alternatively, no less than about 2; alternatively, no less than about 1; or alternatively, no less than about 0.5 mm. The maximum thickness of the absorbent article (40) typically is no greater than about 1; alternatively, no greater than about 2; alternatively, no greater than about 3; alternatively, no greater than about 4; alternatively, no greater than about 5; alternatively, no greater than about 6; alternatively, no greater than about 7; alternatively, no greater than about 8; alternatively, no greater than about 9; or alternatively, no greater than about 10 mm. Thus, the absorbent article (40) may have a thickness of about 10 mm or less; although the approximate thickness of the absorbent article may vary according to, inter alia, the general design and intended disposition of the absorbent article within the vestibule (42) of a female wearer.

The absorbent article (40) typically is folded along an axis lying on or positioned parallel to the principal longitudinal axis (L), as illustrated at least in FIG. 12, prior to disposition within the vestibule (42) of the female wearer. When folded along such an axis, the absorbent article (40) will form a recess (92) which protects the wearer's finger(s) from soiling when the absorbent article is disposed within the vestibule (42). Once inserted, the absorbent article (40) may have a tendency to unfold in an attempt to fill the vestibule and thus maintain the upper surface of the absorbent article in contact with the tissues of the vestibule (42). The absorbent article (40) may be resiliently biased along the axis about which it is folded to increase the tendency of the absorbent article to unfold. Alternatively, the absorbent (66) of the absorbent article (40) may be thicker along its longitudinal edges, as illustrated at least in FIGS. 11 and 12, thus also demonstrating a biasing effect, if desired, which is typically intended to allow the upper surface of the absorbent article (40) to contact the tissues of the vestibule (42). An absorbent article (40) as described herein, however, does not necessarily require any additional features to maintain contact with the tissues of the vestibule (42) of the female wearer. The naturally moist surfaces of the tissues of the vestibule (42) typically demonstrate a tendency to maintain contact with the upper surface of the absorbent article (40).

Although previously described herein as typically being folded along an axis lying on or positioned parallel to the principal longitudinal axis (L), an absorbent article (40) may also be folded along an axis lying on or positioned parallel to the principal transverse axis (T) prior to disposition within the vestibule (42) of the female wearer. When folded along such an axis, the absorbent article (40) typically still forms a recess (92) which protects the wearer's finger(s) from soiling when the absorbent article is disposed within the vestibule (42). Once inserted, the absorbent article (40) may have a tendency to unfold in an attempt to fill the vestibule and thus maintain the upper surface of the absorbent article in contact with the tissues of the vestibule (42). The absorbent article (40) may be resiliently biased along the axis about which it is folded to increase the tendency of the absorbent article to unfold. Alternatively, the absorbent (66) of the absorbent article (40) may be thicker along its transverse end areas (76, 78) thus also demonstrating a biasing effect, if desired, which is typically intended to allow the upper surface of the absorbent article (40) to contact the tissues of the vestibule (42). An absorbent article (40) as described herein, however, does not necessarily require any additional features to maintain contact with the tissues of the vestibule (42) of the female wearer. The naturally moist surfaces of the tissues of the vestibule (42) typically demonstrate a tendency to maintain contact with the upper surface of the absorbent article (40).

Suitable for use with a variety of absorbent articles (40), including the labial pads described herein, is at least one finger-receiving area of dimensions sufficient to receive, at least temporarily, at least a portion of at least one finger of a female wearer. In one embodiment, an absorbent article (40), such as a labial pad, includes an absorbent (66). The absorbent article (40) has a lower surface. The absorbent article (40) is adapted to define a finger-receiving area. The finger-receiving area is of dimensions sufficient to allow the wearer to position at least a portion of at least one finger substantially parallel to a principal axis (i.e., principal longitudinal axis (L) or principal transverse axis (T)) of the absorbent article. The absorbent article (40) is folded substantially about a principal axis to form a recess (92) on the lower surface of the absorbent article. The folded absorbent article (40) typically results in the lower surface of the absorbent article having substantially identical halves facing and opposing each other, as illustrated for example in FIG. 12. In a specific embodiment, the recess (92) is formed by folding the absorbent article (40) substantially about the principal longitudinal axis (L). Situated within the recess (92) and located on the lower surface of the absorbent article (40) is at least one point of juncture (96). The point of juncture (96) serves to at least temporarily join the opposed and facing halves of the lower surface of the absorbent article (40). When so joined, the opposed and facing halves of the lower surface of the absorbent article (40) define the finger-receiving area in a manner that allows at least a distal portion of at least one finger of the wearer to be positioned substantially parallel to the principal longitudinal axis (L). In an alternate specific embodiment, the recess (92) is formed by folding the absorbent article (40) substantially about the principal transverse axis (T). Situated within the recess (92) and located on the lower surface of the absorbent article (40) is at least one point of juncture (96). The point of juncture (96) serves to at least temporarily join the opposed and facing halves of the lower surface of the absorbent article (40). When so joined, the opposed and facing halves of the lower surface of the absorbent article (40) define the finger-receiving area in a manner that allows at least a distal portion of at least one finger of the wearer to be positioned substantially parallel to the principal transverse axis (T).

In a further embodiment, an absorbent article (40), such as a labial pad, includes an absorbent (66) and a liquid impermeable baffle (64). The absorbent article (40) has a lower surface. The baffle (64) forms at least a portion of the lower surface of the absorbent article (40). The absorbent article (40) is adapted to define a finger-receiving area. The finger-receiving area is of dimensions sufficient to allow the wearer to position at least a portion of at least one finger substantially parallel to a principal axis (i.e., principal longitudinal axis (L) or principal transverse axis (T)) of the absorbent article. The absorbent article (40) is folded substantially about a principal axis to form a recess (92) on the lower surface of the absorbent article. The folded absorbent article (40) typically results in the lower surface of the absorbent article having substantially identical halves facing and opposing each other, as illustrated for example in FIG. 12. In a specific further embodiment, the recess (92) is formed by folding the absorbent article (40) substantially about the principal longitudinal axis (L). Situated within the recess (92) and located on the lower surface of the absorbent article (40) is at least one point of juncture (96). The point of juncture (96) serves to at least temporarily join the opposed and facing halves of the lower surface of the absorbent article (40). When so joined, the opposed and facing halves of the lower surface of the absorbent article (40) define the finger-receiving area in a manner that allows at least a distal portion of at least one finger of the wearer to be positioned substantially parallel to the principal longitudinal axis (L). In an alternate specific further embodiment, the recess (92) is formed by folding the absorbent article (40) substantially about the principal transverse axis (T). Situated within the recess (92) and located on the lower surface of the absorbent article (40) is at least one point of juncture (96). The point of juncture (96) serves to at least temporarily join the opposed and facing halves of the lower surface of the absorbent article (40). When so joined, the opposed and facing halves of the lower surface of the absorbent article (40) define the finger-receiving area in a manner that allows at least a distal portion of at least one finger of the wearer to be positioned substantially parallel to the principal transverse axis (T).

In an additional embodiment, an absorbent article (40), such as a labial pad, includes an absorbent (66) and a fluid permeable cover (62). The absorbent article (40) has a lower surface. The cover (62) forms at least a portion of the lower surface of the absorbent article (40). The absorbent article (40) is adapted to define a finger-receiving area. The finger-receiving area is of dimensions sufficient to allow the wearer to position at least a portion of at least one finger substantially parallel to a principal axis (i.e., principal longitudinal axis (L) or principal transverse axis (T)) of the absorbent article. The absorbent article (40) is folded substantially about a principal axis to form a recess (92) on the lower surface of the absorbent article. The folded absorbent article (40) typically results in the lower surface of the absorbent article having substantially identical halves facing and opposing each other, as illustrated for example in FIG. 12. In a specific additional embodiment, the recess (92) is formed by folding the absorbent article (40) substantially about the principal longitudinal axis (L). Situated within the recess (92) and located on the lower surface of the absorbent article (40) is at least one point of juncture (96). The point of juncture (96) serves to at least temporarily join the opposed and facing halves of the lower surface of the absorbent article (40). When so joined, the opposed and facing halves of the lower surface of the absorbent article (40) define the finger-receiving area in a manner that allows at least a distal portion of at least one finger of the wearer to be positioned substantially parallel to the principal longitudinal axis (L). In an alternate specific additional embodiment, the recess (92) is formed by folding the absorbent article (40) substantially about the principal transverse axis (T). Situated within the recess (92) and located on the lower surface of the absorbent article (40) is at least one point of juncture (96). The point of juncture (96) serves to at least temporarily join the opposed and facing halves of the lower surface of the absorbent article (40). When so joined, the opposed and facing halves of the lower surface of the absorbent article (40) define the finger-receiving area in a manner that allows at least a distal portion of at least one finger of the wearer to be positioned substantially parallel to the principal transverse axis (T).

The point of juncture (96) of the various embodiments described herein may be situated in a variety of locations on the lower surface of an absorbent article (40). For example, the point of juncture (96) may be situated in: the central region (74) of both opposed and facing halves; the central region of one opposed and facing half and the first end region (70) of the other opposed and facing half; the central region of one opposed and facing half and the second end region (72) of the other opposed and facing half; the first end region of both opposed and facing halves; the first end region of one opposed and facing half and the second end region of the other opposed and facing half; or the second end region of both opposed and facing halves.

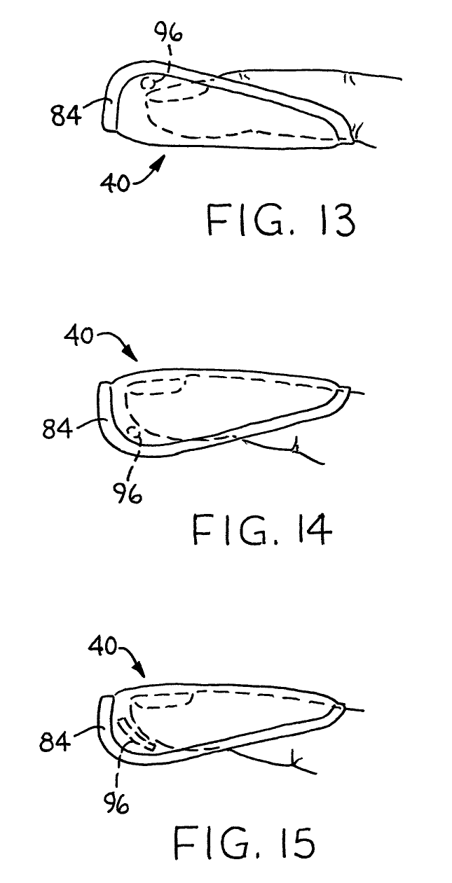

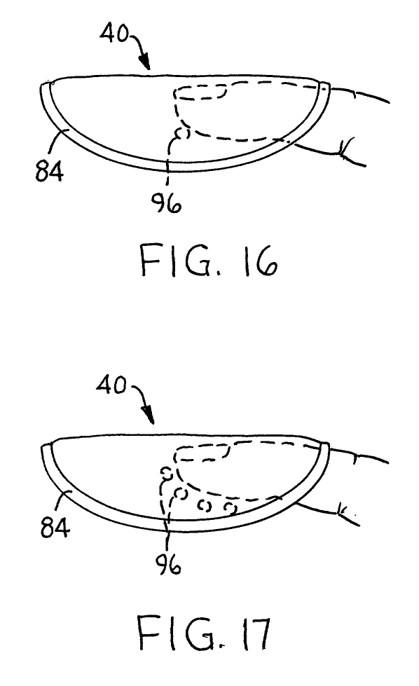

The point of juncture (96) of the present invention may be provided by any of a number of joinder methods known to one of skill and suitable to at least temporarily join the opposed and facing halves of the lower surface of an absorbent article (40) for the purpose of defining the finger-receiving area described herein. One of skill in the art will appreciate that the material(s) situated on the lower surface of an absorbent article (40) will play a significant role in determining a specific suitable joinder method. Methods suitable for joining the opposed and facing halves of the lower surface and providing at least one point of juncture (96) include, by way of example and not by way of limitation, bonding (e.g., ultrasonics, thermal bonding, or the application of adhesives), entangling, fusing, crimping, hot-sealing, embossing, and the like. Moreover, the point of juncture (96) may be a discrete point (as illustrated at least in FIGS. 13, 14 and 16), a series of more than one discrete point (as illustrated at least in FIG. 17) or a continuous point (as illustrated at least in FIG. 15).

The finger-receiving area described herein may be used to assist the female wearer in the disposition of a labial pad into her vestibule (42) while minimizing finger-contact with the upper surface of an absorbent article (40), such as a labial pad. By placing at least a portion of at least one finger in the finger-receiving area prior to insertion, the female user also optimizes the likelihood that her finger(s) will remain unsoiled and the upper surface of the absorbent article (40) will remain in a sanitary condition. In addition, the finger-receiving area also offers a female wearer the opportunity to place at least a portion of at least one finger in a clean or unsoiled location to aid in the removal of an absorbent article (40) and thus minimize the likelihood that the her finger(s) will come into contact with the body-facing surface of the possibly soiled absorbent article.

As various changes could be made in the foregoing finger-receiving areas without departing from the scope of the invention, it is intended that all matter contained in the above description and shown in the accompanying drawings shall be interpreted as illustrative and not in a limiting sense.

What is claimed is:

1. An absorbent article (40) comprising an absorbent (66), the absorbent article being configured for disposition within the vestibule (42) of a female wearer and having a lower surface, the lower surface of the absorbent article being adaptable to define a finger-receiving area of dimensions sufficient to at least temporarily receive at least a portion of at least one finger of the wearer in a manner that allows at least that portion of a finger to be positioned substantially parallel to a principal axis of the absorbent article.

2. The absorbent article of claim 1, wherein the absorbent article further comprises a recess (92), the recess formed by folding the absorbent article substantially about a principal axis, such folding resulting in the lower surface of the absorbent article having substantially identical halves facing and opposing each other.

3. The absorbent article of claim 2, wherein the absorbent article further comprises a principal longitudinal axis (L) and a principal transverse axis (T).

4. The absorbent article of claim 3, the recess having been formed by folding the absorbent article substantially about the principal longitudinal axis, the absorbent article further comprising first (70) and second (72) end regions and a central region (74) disposed between the first and second end regions, and at least one point of juncture (96) situated within the recess and located on the lower surface of the absorbent article, the point of juncture at least temporarily joining the opposed and facing halves to define the finger-receiving area in a manner that allows at least a distal portion of at least one finger to be positioned substantially parallel to the principal longitudinal axis.

5. The absorbent article of claim 4, wherein the point of juncture is located on that portion of the lower surface situated in at least one of the end regions of the absorbent article.

6. The absorbent article of claim 5, wherein the point of juncture comprises a discrete point.

7. The absorbent article of claim 5, wherein the point of juncture comprises a continuous point.

8. The absorbent article of claim 4, wherein the point of juncture is located on that portion of the lower surface situated in at least the central region of the absorbent article.

9. The absorbent article of claim 8, wherein the point of juncture comprises a discrete point.

10. The absorbent article of claim 8, wherein the point of juncture comprises a continuous point.

11. The absorbent article of claim 3, the recess having been formed by folding the absorbent article substantially about the principal transverse axis, the absorbent article further comprising first (70) and second (72) end regions and a central region (74) disposed between the first and second end regions, and at least one point of juncture (96) situated within the recess and located on the lower surface of the absorbent article, the point of juncture at least temporarily joining the opposed and facing halves to define the finger-receiving area in a manner that allows at least a distal portion of at least one finger to be positioned substantially parallel to the principal transverse axis.

12. The absorbent article of claim 11, wherein the point of juncture is located on that portion of the lower surface situated in at least one of the end regions of the absorbent article.

13. The absorbent article of claim 12, wherein the point of juncture comprises a discrete point.

14. The absorbent article of claim 12, wherein the point of juncture comprises a continuous point.

15. The absorbent article of claim 11, wherein the point of juncture is located on that portion of the lower surface situated in at least the central region of the absorbent article.

16. The absorbent article of claim 15, wherein the point of juncture comprises a discrete point.

17. The absorbent article of claim 15, wherein the point of juncture comprises a continuous point.

18. An absorbent article (40) comprising an absorbent (66) and a liquid impermeable baffle (64), the absorbent article being configured for disposition within the vestibule (42) of a female wearer and having a lower surface, the baffle forming at least a portion of the lower surface of the absorbent article, the lower surface of the absorbent article being adaptable to define a finger-receiving area of dimensions sufficient to at least temporarily receive at least a portion of at least one finger of the wearer in a manner that allows at least that portion of a finger to be positioned substantially parallel to a principal axis of the absorbent article.

19. The absorbent article of claim 18, wherein the absorbent article further comprises a recess (92), the recess formed by folding the absorbent article substantially about a principal axis, such folding resulting in the lower surface of the absorbent article having substantially identical halves facing and opposing each other.

20. The absorbent article of claim 19, wherein the absorbent article further comprises a principal longitudinal axis (L) and a principal transverse axis (T).

21. The absorbent article of claim 20, the recess having been formed by folding the absorbent article substantially about the principal longitudinal axis, the absorbent article further comprising first (70) and second (72) end regions and a central region (74) disposed between the first and second end regions, and at least one point of juncture (96) situated within the recess and located on the lower surface of the absorbent article, the point of juncture at least temporarily joining the opposed and facing halves to define the finger-receiving area in a manner that allows al least a distal portion of at least one finger to be positioned substantially parallel to the principal longitudinal axis.

22. The absorbent article of claim 21, wherein the point of juncture is located on that portion of the lower surface situated in at least one of the end regions of the absorbent article.

23. The absorbent article of claim 22, wherein the point of juncture comprises a discrete point.

24. The absorbent article of claim 22, wherein the point of juncture comprises a continuous point.

25. The absorbent article of claim 21, wherein the point of juncture is located on that portion of the lower surface situated in at least the central region of the absorbent article.

26. The absorbent article of claim 25, wherein the point of juncture comprises a discrete point.

27. The absorbent article of claim 25, wherein the point of juncture comprises a continuous point.

28. The absorbent article of claim 20, the recess having been formed by folding the absorbent article substantially about the principal transverse axis, the absorbent article further comprising first (70) and second (72) end regions and a central region (74) disposed between the first and second end regions, and at least one point of juncture (96) situated within the recess and located on the lower surface of the absorbent article, the point of juncture at least temporarily joining the opposed and facing halves to define the finger-receiving area in a manner that allows at least a distal portion of at least one finger to be positioned substantially parallel to the principal transverse axis.

29. The absorbent article of claim 28, wherein the point of juncture is located on that portion of the lower surface situated in at least one of the end regions of the absorbent article.

30. The absorbent article of claim 29, wherein the point of juncture comprises a discrete point.

31. The absorbent article of claim 29, wherein the point of juncture comprises a continuous point.

32. The absorbent article of claim 28, wherein the point of juncture is located on that portion of the lower surface situated in at least the central region of the absorbent article.

33. The absorbent article of claim 32, wherein the point of juncture comprises a discrete point.

34. The absorbent article of claim 32, wherein the point of juncture comprises a continuous point.

35. An absorbent article (40) comprising an absorbent (66) and a fluid permeable cover (62), the absorbent article being configured for disposition within the vestibule (42) of a female wearer and having a lower surface, the cover forming at least a portion of at least the lower surface of the absorbent article, the lower surface of the absorbent article being adaptable to define a finger-receiving area of dimensions sufficient to at least temporarily receive at least a portion of at least one finger of the wearer in a manner that allows at least that portion of a finger to be positioned substantially parallel to a principal axis of the absorbent article.

36. The absorbent article of claim 35, wherein the absorbent article further comprises a recess (92), the recess formed by folding the absorbent article substantially about a principal axis, such folding resulting in the lower surface of the absorbent article having substantially identical halves facing and opposing each other.

37. The absorbent article of claim 36, wherein the absorbent article further comprises a principal longitudinal axis (L) and a principal transverse axis (T).

38. The absorbent article of claim 37, the recess having been formed by folding the absorbent article substantially about the principal longitudinal axis, the absorbent article further comprising first (70) and second (72) end regions and a central region (74) disposed between the first and second end regions, and at least one point of juncture (96) situated within the recess and located on the lower surface of the absorbent article, the point of juncture at least temporarily joining the opposed and facing halves to define the finger-receiving area in a manner that allows at least a distal portion of at least one finger to be positioned substantially parallel to the principal longitudinal axis.

39. The absorbent article of claim 38, wherein the point of juncture is located on that portion of the lower surface situated in at least one of the end regions of the absorbent article.

40. The absorbent article of claim 39, wherein the point of juncture comprises a discrete point.

41. The absorbent article of claim 39, wherein the point of juncture comprises a continuous point.

42. The absorbent article of claim 38, wherein the point of juncture is located on that portion of the lower surface situated in at least the central region of the absorbent article.

43. The absorbent article of claim 42, wherein the point of juncture comprises a discrete point.

44. The absorbent article of claim 42, wherein the point of juncture comprises a continuous point.

45. The absorbent article of claim 37, the recess having been formed by folding the absorbent article substantially about the principal transverse axis, the absorbent article further comprising first (70) and second (72) end regions and a central region (74) disposed between the first and second end regions, and at least one point of juncture (96) situated within the recess and located on the lower surface of the absorbent article, the point of juncture at least temporarily joining the opposed and facing halves to define the finger-receiving area in a manner that allows at least a distal portion of at least one finger to be positioned substantially parallel to the principal transverse axis.

46. The absorbent article of claim 45, wherein the point of juncture is located on that portion of the lower surface situated in at least one of the end regions of the absorbent article.

47. The absorbent article of claim 46, wherein the point of juncture comprises a discrete point.

48. The absorbent article of claim 46, wherein the point of juncture comprises a continuous point.

49. The absorbent article of claim 45, wherein the point of juncture is located on that portion of the lower surface situated in at least the central region of the absorbent article.

50. The absorbent article of claim 49, wherein the point of juncture comprises a discrete point.

51. The absorbent article of claim 49, wherein the point of juncture comprises a continuous point.
